(19) United States
(12) Patent Application Publication
(10) Pub. No.: US 2005/0149145 A1
(43) Pub. Date: Jul. 7, 2005

(54) Enhanced device for diminishing or eliminating the pain caused by superficial therapeutic injection or superficial body tissue sampling or the pain from a superficial injury as well as for the reduction of hemorrhage from an injured area

(76) Inventor: George Gary Coulter, Nashville, IN (US)

Correspondence Address: BAKER & DANIELS, 300 NORTH MERIDIAN STREET, SUITE 2700, INDIANAPOLIS, IN, 46204-1782, US

(21) Appl. No.: 10/746,685

(22) Filed: Dec. 29, 2003

(51) Int. Cl. A61N 1/18

(52) U.S. Cl. 607/46

(57) ABSTRACT

A device is disclosed which couples the use of Trans Epithelial Nerve Stimulation with the administration of a therapeutic injection, such as an immunization, or tissue sampling procedure such as deriving a blood sample. By such an arrangement the discomfort associated with these procedures may be considerably reduced or eliminated, thereby improving compliance with a range of medical procedures. To date TENS has not been used in such an arrangement, rather development in TENS technology has been aimed at improving its efficacy. Similarly injection technology developments have been orientated around reducing cross infection risks and accidental injury to operator or patient.

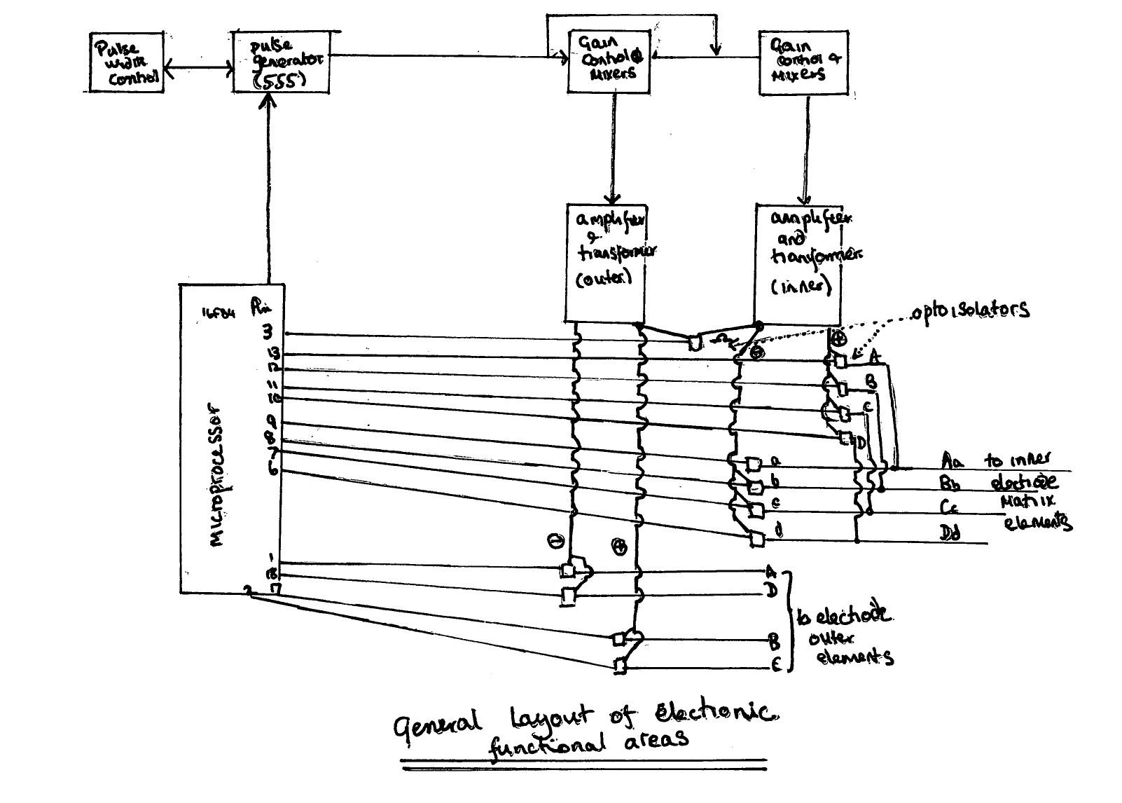

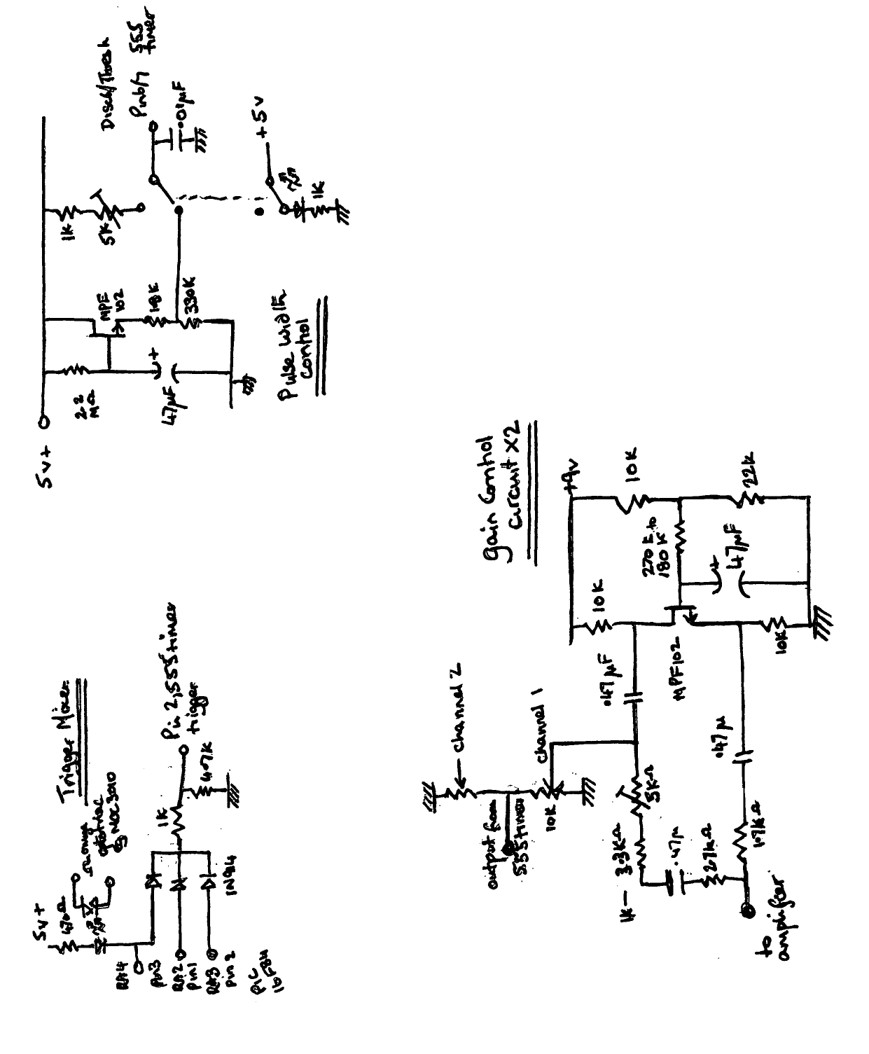

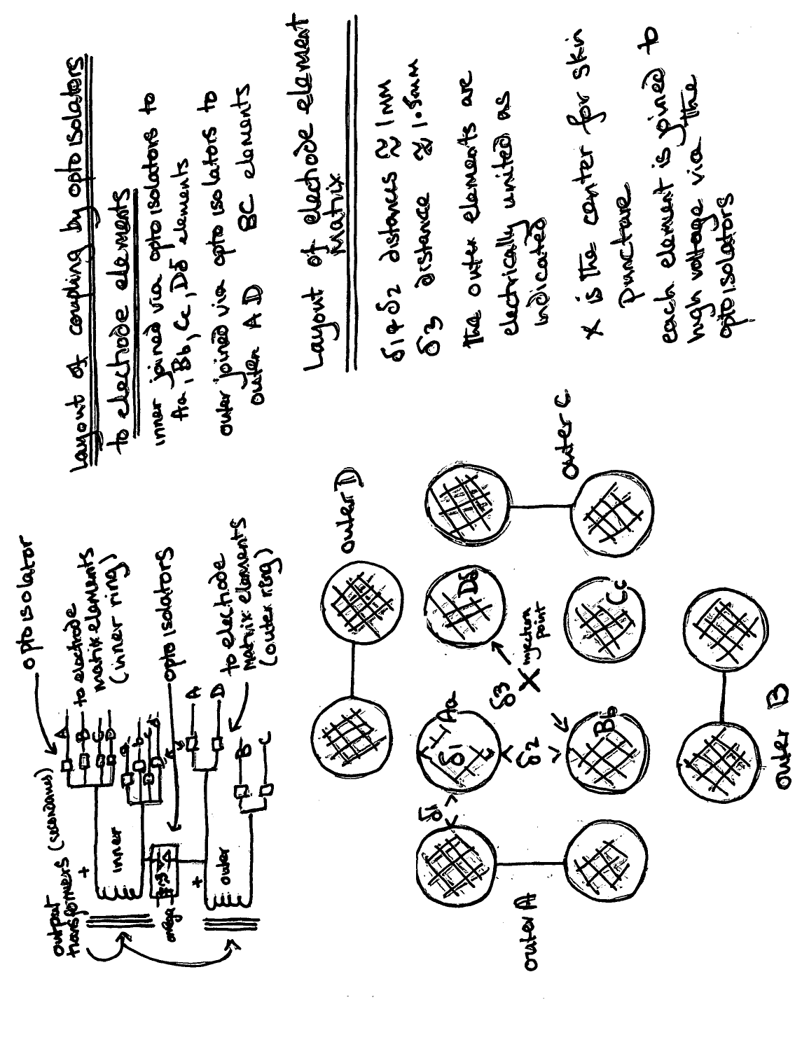

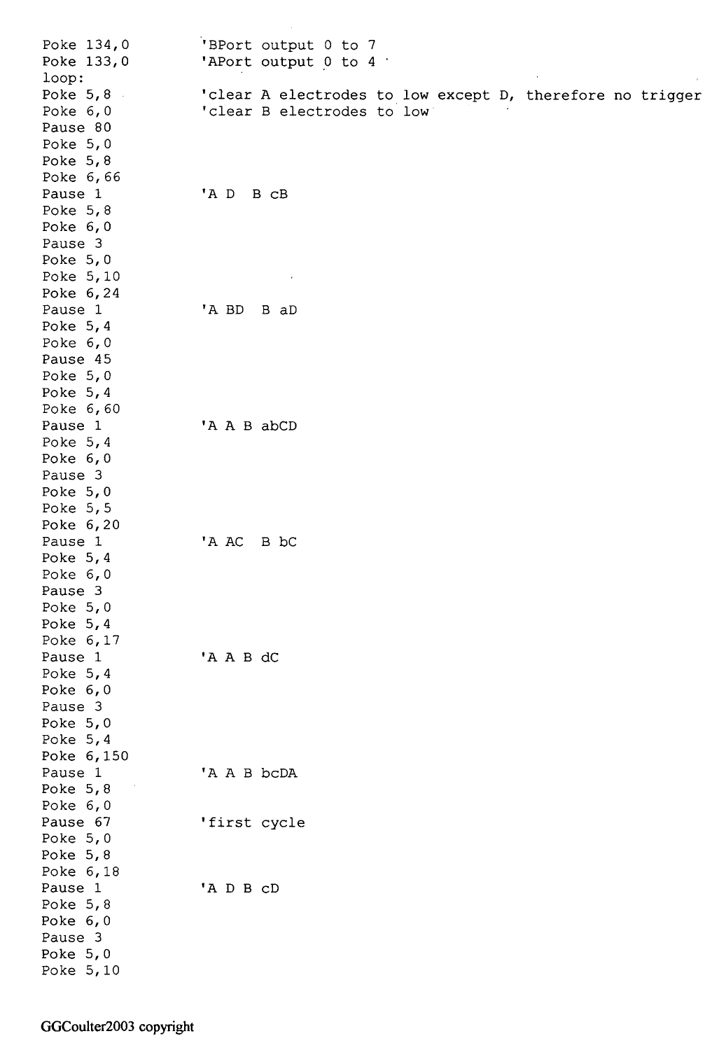

### CROSS REFERENCE TO RELATED APPLICATIONS

Application Ser. No. 10/195,171

### STATEMENT REGARDING FEDERALLY SPONSORED RESEARCH OR DEVELOPMENT

Not Applicable

### REFERENCE TO A MICROFICHE APPENDIX

Not Applicable

### BACKGROUND OF THE INVENTION

Technology developments in the field of effecting a therapeutic injection, such as an immunization, or off a sampling of a body tissue, such as blood, have predominantly been orientated around the avoidance of cross contamination, accuracy of injection or avoidance of operator injury. Some diminution of pain of the recipient has often been a secondary consideration and has proved a somewhat intractable goal. This is at least in part due to some off the factors defining the likelihood and extent off pain are related to the nature of substance to be injected, site and depth of injection and the psychological expectation of pain by the patient. For the most part the common injections performed are regarded as inducing only minor and transient pain or discomfort. However, despite the modest level of discomfort associated with many common injections, needle phobia and the discomfort of injection remain as significant barriers to many therapies. For example, regimens for diabetes control are sometimes not adhered to because of the discomfort inherent in blood glucose testing and or insulin injection. Partial amelioration of these concerns and effects can be achieved by the use of the device described in filing application Ser. No. 10/195,171. However, a more complete elimination of coherent pain signaling, as well as an improved means of disorganizing and fragmenting unblocked pain signals that are induced on tissue injury, can be achieved by the novel enhancements described below. In addition the device may be adapted so as to be used during other procedures where the skin is breached, such as the insertion of an intravascular canula or other medical device such as a pharmaceutical slow release capsule. Further, this system may be modified such that the electrode surface may be incorporated into a surgical/medical dressing allowing for the application of the electrical signals with or without an additional vibration element. This would allow a local soothing or analgesic effect on a wound and in addition, by means of causing vascular smooth muscle contraction, control local bleeding.

### SUMMARY OF INVENTION

This invention provides a means of reducing or eliminating pain on skin breach such as during a therapeutic injection, by the application of TENS (trans-epithelial nerve stimulation) with or without a co stimulus of vibration before during and after an injection. The method is also applicable for superficial pain relief or hemorrhage as may be useful in the management of a surgical or other wounds. The beneficial effect is achieved through the use of an electrode array, which meets certain defined specifications in the size and arrangement of electrode elements, in association with a control unit, that switches TENS signals between these various electrode elements in a specified manner. By this means the locality of the TENS current can be controlled and differing nerve fibers can be independently targeted. The pattern of electrode activity is such that the total exposure area may have different signal patterns applied within different regions of this total exposure area. In general an inner area (proximal to the site of skin breach) has an applied pattern of TENS activity of a regular or more usually an irregular spacing between pulses, both in time and the specific route of electrical discharge. The time interval between individual pulses in this electrode area is generally a matter of a few milliseconds (though may be longer), whereas full coverage of the anatomy of this inner electrode area by a TENS signal takes a few tenths or hundredths of seconds. A second area, more peripheral to the site of injection or tissue damage, has a pulse pattern which may be regular or more usually irregular with inter-pulse pauses that may vary up to a few tenths of a second. This latter area is less demanding in the specifications required of TENS application, excepting the pulse interval pattern. The latter pattern of pulses may be added to or superimposed on the inner electrode elements in addition to their own blocking pattern, but a higher intensity is required where the two signals overlap. The effect of the outer element signal pattern, or its overlay on the inner electrode elements, is such that a sensation of irregular or patterned buzzing or prickling is noted by the recipient. This sharp irregular sensation makes it difficult or impossible to recognize the timing of the sharp sensation that is produced when a sharp instrument (needle) contacts the skin. This sensation is one of the key elements in the normal perception of pain during an injection and its disruption contributes to the efficacy of this system. The two electrode areas may intermittently have pulses applied between individual electrode elements of each area, though for the most part they are independently active and thus may both be energized as separate circuits at the same time. When both areas are energized at the same time in effect the overall signal strength may be double at that instant. The current general designs of the signal application electrodes used for TENS as well as the type of TENS generating and control units are inappropriate for this end. Rather they are better adapted to wider and deeper signal penetration utilizing electrodes of a larger area and with fewer electrode elements capable of being exposed to different signals. The preferred electrodes are arranged such that the gaps or spacing between opposite conducting electrode edges in the region of skin puncture are kept within an order of magnitude of the dimensions of skin (epidermal thickness usually being about 0.2 millimeters). Thus the electrode to electrode elements gap in the region of and adjacent to skin puncture is expected to be of the order of a millimeter or so, except where needle dimensions allow for less, or require a greater gap, such as for an intra-vascular cannula insertion. In the situation where a larger space is required for the injection (for example a cannula insertion) inter-electrode gap is kept to a minimum by employing additional electrode elements wherever possible within the inner encircling ring that circumscribes the area of skin puncture. This inner electrode arrangement affords more anatomically controlled and exact local TENS exposure of the epidermal and dermal nerve fibers, particularly C fibers which have terminals located in the epidermis as well as in deeper layers. In addition the deeper sited nerve fibers may be interacted with by these electrodes and the more widely spaced electrode combinations, switching of the TENS signal between individual and multiple electrode surfaces of the overall electrode matrix in rapid succession. Further, in addition to the gap between electrode elements, individual electrode element surface area is small (2-5 square millimeters or less), particularly those most proximal to the injection site. This further aids the localization of current flow, reducing the tendency for deeper and wider tissue stimulation as may be seen by the muscle stimulation that occurs with prevalent electrode arrangements with larger surface areas active at any one instant. The total area of tissue exposure is widened by switching activity in electrode pairs in a rapid sequential manner across the total electrode matrix area. By so doing, the area active at any one instant is small yet coverage of the total area requiring TENS exposure is effected. The TENS signals are applied across this array of electrodes and are varied in polarity, voltage, frequency, wave form and timing of application.

Different anatomical locations on the body require different settings for maximum effect and comfort. Within the locale of the electrode matrix, different electrode pairs or combinations may be concurrently targeted for either nerve blockade or counter irritation nerve stimulation causing a distraction signal to the patient. In areas less proximal to the site of skin puncture, the objective of TENS application is to produce counter irritation or a distracting signal, blockade being a secondary objective. The peripheral (distracting) signal also allows the centrally located electrode surfaces to deliver a higher strength of signal than would otherwise be comfortable, to a degree masking the more tonic sensation created by the signals applied to these electrode elements. The outer element function requires a less exacting specification for current application depth and therefore electrode size and geometry may be larger and more separated. The TENS signal waveform applied may be a standard repetitive uni- or bipolar pulse (either in a prolonged series or in bursts), as in the ordinary use of TENS. Mote complex wave forms may be used, such as described in U.S. Pat. No. 4,723,552. This latter may allow for the tuning of the TENS current to the characteristics of the nerve and nerve endings targeted, reducing the tendency of spread and allowing a lower power TENS signal to be effective. However, with the present arrangement a simple unidirectional pulse is effective, when applied in the fashion described below, particularly noting the frequency and irregularity off pulse intervals between pulses as they are applied through the two electrode areas. The provision of a concurrent physical vibration to the device and/or skin both reduces the direct force and energy required for needle penetration (which is related to the perception of pain severity) as well as providing an additional distracting stimulus to the patient and consequent masking of pain perception. Using the same principles of an inner and outer electrode element matrix the electrode may be fashioned into a wound dressing or the electrode array may be fashioned so as to allow biopsy or other surgical device use. In these cases and uses, the smaller and more closely spaced electrode elements are most proximal to the area of tissue injury. Further, as a consequence of vascular smooth muscle contraction resulting either directly or through reflex to the local application of an intense TENS current, hemorrhage my be diminished or eliminated.

### BRIEF DESCRIPTION OF DRAWINGS

Depicted in the 3 pages of drawings is one arrangement of the elements needed for the device to work. In the present depiction, the main elements described in Ser. No. 10/195,171 remain but are modified as described. The specific design of the individual parts may change, the present depiction is intended to represent a possible working execution of the invention by someone skilled in the art of electronics using the parameters described. Attention is drawn to the following areas of the drawings.

Page 1 A block layout of the circuitry elements (excepting power supply) and their relationships for a TENS generating device, microcomputer control and electronic switch matrix comprising the necessary circuitry. The original pulse may be taken directly from the microprocessor or as suggested here it is generated by a standard 555 timing chip configured as a pulse generator and triggered by outputs from the microprocessor. The 555 timer is triggered when one or more suitably connected microprocessor outputs (three in the present embodiment) come to zero, any one being high prevents triggering. A circuit to achieve this end is shown on Page 2 area labeled Trigger Mixer. Attention is drawn to the fact that output A4 (pin 3) on the PIC 16F84 is an open collector output and therefore requires a pull up resistor in order to come high. Advantage of this is taken in this circuit, connecting the output to the voltage supply to this area of circuit (5 volts) via a resistor and through the diode of RA4s' associated optoisolator. The outputs of the three A port outputs used here (RA2,3,4) are fed via signal diodes into a resistor ladder and then to the trigger pin of a 555 timer chip. The pulse width generated by the 555 timer is controlled by a resistor capacitor combination. Attention is drawn here to page 2 area labeled Pulse Width Control. A two way switch connects either a standard variable resistor to pins 6 or 7 (as is the standard method of pulse width control) or to the circuit depicted which uses the variable transconductance of an FET modulated by an RC combination joined to the FETs gate. By this means the pulse width can be caused to be initially long and then decline to a shorter length over a few seconds. The output of the timer is split as depicted also on page 2 area labeled Gain Control Circuit. Each channel has a bypass route through a resistor to the input of the channel amplifier or the signal runs through an FET circuit. The FETs transconductance is controlled by the RC and voltage divider circuit, resulting in an initial signal at the amplifiers input which increases in strength over a few seconds. Each channel can have a different time constant (governed by the RC combination) thereby allowing a gentle and comfortable build up of strong signals eventually reaching the patient.

Page 3 Layout of Coupling Optoisolators to Electrode Elements depicts the arrangement whereby the two output transformers may be joined by an optothyristor. As can be seen, the outputs of the transformers are joined through optothyristors to the electrode elements. Because the signal is unipolar (the output of the transformer may be beneficially protected with a kick back diode) it is possible to control the electrical status and polarity of each electrode element individually. For the inner set of elements the positive terminal is joined via optothyristors to the electrode elements labeled A to D, whereas for the negative terminal is joined to the same four elements by another set of optothyristors. To help distinguish whether the element is brought positive or negative by the microprocessor program, in the case of the negative the electrode elements are labeled a to d (lower case) and A to D (upper case) for a positive state. Page 3 Layout of Electrode Element Matrix, a not to scale diagram is shown to illustrate the relationships of the patient contact surfaces or elements of the electrode matrix. The electrodes conducting material may be metal or a conducting gel as described in U.S. Pat. No. 6,564,079, B1 backed onto a suitable insulating material and may be disposable or capable of being recycled and reused. It is preferable if the patient side of the electrode has a sticky or adherent nature so that the electrode may be stuck to the patients' skin during use. Each element is electrically isolated from its fellows though the outer elements may be joined in pairs. The array depicted indicates one of many possible tesallated arrangements of the individual electrode contact surface shapes. The common points to these various arrays are the individual electrode element area and inter electrode gap (as described above) in the region of the needle hole or skin breach area, less exacting arrangements being possible further away from the needle's point of entry. The dorsum of the electrode affords an insulated wiring surface that connects through to the ventral surface allowing suitable electrical connections to be made and routed via a suitable connector and then to the TENS device described above. Attention is drawn to a number of features. First x marks the point where skin penetration occurs and is surrounded by four (in this case) electrical surfaces that are each less than 5 and preferably less than 4 square millimeters in area. In the case of circular surfaces to these elements d1 equals d2 and is about 1 millimeter in length. D3 in all cases is minimized to be large enough to accommodate the needle diameter, but where this distance needs to be greater than 2 millimeters the addition of further electrode elements in the inner circle may add flexibility in programming and reliability. By activating pairs of optoisolators, connections between the various electrode elements and the poles of the output transformers are made. A ring of discharge pathways through the patients skin may be sequentially created by activating electrode element pairs around the point of penetration (x) giving good coverage of the sensory nerves to the area. The outer electrode elements (labeled outer A through to D) do not need to be capable of attachment to both positive and negative terminals, rather discharge is sufficient for example if A or D is positive with B or C being negative. Further the geometry and area for these elements is less critical. They may be composed of two or more elements (similar to the inner circle of elements as here) joined together as indicated or some other geometrical arrangement may be created. With the elements arranged as depicted an optoisolator may join the two output transformers of the TENS device above. A connection between the positive terminal of the transformer serving the inner electrodes and the negative end of the transformer serving the outer elements may be made with an optoisolator (omega). In this arrangement the inner element may be activated in association with an outer element and will always be the negative pole (e.g. inner A to outer B results from optoisolators inner A outer B and omega being on). This enhances the ability to keep the inner area covered with a depolarizing current. Alternatively reversing the connections between the output transformers allows an opportunity to make all outer elements including A and D serve as negative poles.

The output circuitry is so arranged as to give a pulse voltage up to 360 volts from the output side of the output transformer. There are multiple ways of executing the output elements, the use of an output transistor such as MJE3055T, biased to class B or C operation, with the transformer primary being the collector load and a having turns ratio of 1:44, with a supply voltage of 9 volts, works well. Largely the primary impedance defines the need for the power rating of the output circuitry, a higher primary impedance allows for lower power output units. The microprocessor employed may be a number of models such as those of the BASIC STAMP series or of the PIC micro series. In this embodiment the PIC 16F84, or its one time programmable equivalent, has been used with success. This all may be housed in the setting as described in patent application Ser. No. 10/195,171, but more usually would be housed in a suitable container such that either the patient or administering caregiver may control the various settings and levels. The output of the device in this case would be via a multi-cored cable to a suitable electrode as described.

Page 4 A page of layout of computer code suitable for the PIC 16F84 illustrating the key elements of electrode switching. Attention is drawn to setting the RAB and RAA ports to output and then switching combinations of these so activating the optothyristors controlling the electrode elements. Note the form of various pauses between operation in particular the delay between activations to fully turn off the optothyristors. The PIC Micro may be operated up to 20 MHz.

### DETAILED DESCRIPTION OF THE INVENTION

The over arching strategy employed in this invention rests on two main insights. First the current embodiments of TENS device are not optimized for the anatomical location of nerves within the skin or the local intensity of exposure required for significant local blockade.

Secondly, nature builds in a great deal of redundancy in all its systems including to communication. For example in written communication the well known sentence “if u cn rd the msg . . . ” clearly indicates the redundancy of information communicated in a correctly spelled sentence. The same strategy of reliability through the use of redundancy is true of the various biological systems of the body with generally over capacity of each system and adjunctive but none essential activity in each system. Thus the same message sent by multiple routes (nerve fibers) can accommodate some noise in each route but the combined central message arrives adequate for clear interpretation. In the peripheral nervous system this generally ensures more certain communication, in the same way as “packeting” of information within a telephone system, with multiple routes taken by the packets ensuring more noise free and certain delivery of the message. With approximately 670 neurites or more per square millimeter of the skin of the finger tips, yet a two point discrimination ability of around two millimeters there appears to be massive in-built redundancy. Translating the implications of these insights into an effort of controlling the peripheral nervous system, suggests that completely blocking the nervous impulses generated on tissue injury in a given area (such as the site of injection) is unlikely to be fully effective, unless exceedingly strong means are employed. Rather turning general communication down but also adding dis-information into the communication pathways is more likely to and in fact does work, with poorly interpretable or un-interpretable signals reaching the Central Nervous System from the periphery. Thus the system described is designed to improve local delivery of TENS to a specific area and to add signals that disrupt interpretation of remaining signals by adding counterfeit or noise signals to those nerves still operating at a biologically adequate level. By this means the Central Nervous System does not receive enough consistent information to correctly register the signals of skin breach and tissue injury. Specifically, firstly the electrode layout and switching activity between electrode pairs increases local TENS exposure proximal to the injection site, affording an optimized level of local blockade incomplete as it is liable to be. Secondly the irregular or difficult to interpret timing pattern of strong pulses applied to the outer elements or on the background of more frequent (blocking) pulses makes it difficult for the patient to recognize the timing and sharpness of skin penetration. By this means the normal neuronal activity of skin breach is blocked and masked by the background of disrupting signals and information.

Efforts have been conducted in the general development of TENS elucidating various parameters of pulse size, pulse width, form, number and spacing of pulses in a pulse trains and electrode contact design. At best these allow for optimizing this weak form of pain control for deeper tissues and for the most part they have a poor record of success. Many devices are in the public domain and are generally used in the management of chronic pain. In the case where TENS has been proposed for acute use, it is noted that the application is clearly intended as an alternative or adjunct to the use of a pharmacological anesthetic when a surgical intervention is envisaged and is intended to operate for some minutes (examples U.S. Pat. Nos. 6,351,674B2, 4,924,880, 5,052,391). With the exception of application Ser. No. 10/195,171 no TENS device has been considered explicitly for the avoidance of the pain expected and induced by a short procedure such as an injection (where indeed the agent being injected may be a local anesthetic). Equally limited success has been seen with manipulating the mechanics of injection, such as needleless injector technology, or refinement of the needle systems into automatic or triggered devices. The principal strategy employed by these designs is to minimize trauma and or “catch the patient unawares”. These have been proposed for either injection or for the collection of small blood samples for example in the assessment of diabetes (examples U.S. Pat. Nos. 6,135,979, 6,102,896, 6,083,197, 5,993,412, 5,746,714). Despite these efforts, pain on therapeutic injection or sampling remains an ongoing and potentially avoidable issue. In the case of current TENS devices of particular note, the various designs of the current application electrodes attempt to maximize general contact area and conductivity thereby improving effectiveness of current transfer to the body part. This may be desirable where a general counter irritation, muscle stimulation or other deep tissue stimulation is a targeted object of TENS application. However, where a specific localized superficial blockade is desired and includes targeting of intra epithelial and dermal nerves, the usual large area electrode arrangement would be inappropriate as good control of the anatomy of current flow is lost. Were the TENS signal to be applied by a needle as one pole of the current application, the same issues arise unless opposite electrodes are applied with a gap between them of about a millimeter and there are multiple conduction pathways. By reducing electrode contact area and inter-electrode gap, control is obtained of the anatomy of current flow, in particular where the skin acts as a dielectric conductor and breaks down its resistance under the electric field. This allows a surety of anatomical nerve or nerve branch targeting, impossible with the prevalent TENS electrode designs. Thus in this proposed device there are two distinct areas of the electrode design intended, one intended for more usual TENS counter irritation application, surrounding the other for a superficial nerve blockade. Further when skin penetration occurs the possible tendency for the TENS signal to “short circuit” (through the needle track in the epidermis) can be accommodated by affording switching of the signals between the inner sited electrode contacts and or those more peripherally located. By this means, the TENS current is relatively increased within the poorly conducting outer layers of the skin (specifically the epidermis) compared to the more dominant usual route whereby current tracks through sweat glands or hair follicles, down to the lower electrical resistance areas of the dermis. Increasing electrode area reduces the electrode to sub-dermal tissue resistance (which in absolute terms with normal sized electrodes is several orders of magnitude lower than a purely epidermal pathway resistance between electrodes). This is because far more sweat glands or hair follicles are covered by the electrode surface. As inter-electrode distance approaches or is less than twice epidermal thickness, conduction along the epidermal tissue becomes significant in proportion to the total current flow, and electrode area reduction further improves this efficiency at the expense of electrode effectiveness. However, the applied voltage needed to cause this current to flow increases, thus a balance is struck between area, voltage and inter-electrode gap such that both epidermal and dermal nerves are adequately exposed to the TENS signal. The arrangement proposed allows the un-myelinated “C” nerve fiber endings that lie within the epidermis and are part of the pain sensing mechanism better exposure to the depolarizing current. In the dermis it is believed that other nerve fiber types may in addition conduct pain signals, particularly the A-delta fibers. There are multiple low resistance routes for the current to take both within and below the dermis that avoid flow around nerve endings, limiting the distance between electrodes and electrode surface areas, increases the electrical potential or gradient in a small area. This both increases the probability of a nerve fiber in this region being depolarized or hyper-polarized and decreases the probability of current tracking into deeper tissues causing side effects such as muscle stimulation. Further, limitation of electrode area decreases electrode to muscle capacitance, thereby reducing signal conduction by capacitative coupling to this tissue and so reducing muscle stimulation.

In the usual application of TENS, acclimatization to the TENS current occurs in respect to discomfort caused by the current. This tolerance to the TENS current evolves in the patient over a short period of time (particularly with a continuous pulse signal as opposed to bursts off pulses at a low frequency). Thus, if the signal applied to the electrodes is built up slowly (over a few seconds) significantly higher signal strengths may be applied without causing significant discomfort. In the working device described here, electrical circuitry is so arranged that the TENS signal(s) builds up in intensity over a few seconds. In addition circuitry is provided that reduces the pulse width of the signal over a few seconds, again allowing a higher voltage to be applied without discomfort. This combination of electrode surface limitation, inter-electrode gap limitation, signal strength build up, pulse width control, outer electrode use and interplay with the inner electrodes as well as the timing and pattern (both in time and locality) of TENS signal application allow the use of an intense local TENS effect. This results in blocking and or distorting the usual pain signal formation and pattern of firing of the local nerves in the area of skin penetration. The overall effect is to cause miscommunication and misinterpretation of the pain signals at higher levels, in effect blocking or drastically reducing the sensation of pain experienced by the local tissue trauma. Each of these elements contributes in part to the overall effect, resulting in a relevant level of efficacy and comfort.

Nerve fiber parameters, frequency and wave form selection.

The possible frequency of action potentials of nerve fibers conducting pain signals may be measured or estimated and from this a projection of the ideal blocking signal parameters. Such information is available to a degree in standard texts of physiology. However, it is likely that there are both a range off fiber types and dimensions as well as their possible branching and networking arrangements that need to be addressed by the TENS signal. In some circumstances as nerve branches coalesce into the main trunk for example the frequency of transmitted impulses decline, in effect acting as a low pass filter. Further, it is known that there is not a simple one to one relationship between frequency of discharge, number of nerves involved or of nerve type and the perceived sensation of pain severity. These observations are consistent with a system with redundancy as described above. Electrically the skin acts as both a resistive and a capacitive barrier in parallel, thus increasing frequency naturally allows a lower resistance to current flow. Higher frequencies tend to direct the signal to muscle which itself has a relative high surface area capacitive quotient than nerve by a factor of 10 or more as well as having more total surface area. Thus, the strategy taken here is to present a sweep of pulses and amplitudes that fit a range of possibilities. TENS is usually applied as a core depolarizing pulse of the order of 50 to 200 microseconds long repetitively applied in a train, which may be broken up into blocks or bursts of pulses. A shorter pulse width (30 to 150 microseconds) appears to have an adequate level of effectiveness, though longer pulses are common in studies on nerve and are more effective in nerve stimulation. Applying these various signal forms through the electrode described produces the desired effect. If a prolonged exposure is desired (such as with a wound dressing) modulation of the plateau level of signal with a time constant of the order of a few tenths to hundredths of a second so helps ensure continuing efficacy. Physical Vibration Application. The perception of the severity of pain is related amongst other things to the force or energy of injury (Differential ability of human cutaneous nociceptors to signal mechanical pain and to produce vasodilatation. Koltzenburg, Handwerker, J Neurosci 1994 March; 14 (3 Pt 2):1756-65). Vibration lowers the resistance to a needle entering the skin (in the same manner that wiggling the fingers in sand allows them to penetrate the sand more easily). In addition the provision of a vibration during the time of injection adds another distracting element to the nervous system. Thus a vibration generating device is added to the device enhancing its effectiveness. ongoing and potentially avoidable issue. In the case of current TENS devices of particular note, the various designs of the current application electrodes attempt to maximize general contact area and conductivity thereby improving effectiveness of current transfer to the body part. This may be desirable where a general counter irritation, muscle stimulation or other deep tissue stimulation is a targeted object of TENS application. However, where a specific localized superficial blockade is desired and includes targeting of intra epithelial and dermal nerves, the usual large area electrode arrangement would be inappropriate as good control of the anatomy of current flow is lost. Were the TENS signal to be applied by a needle as one pole of the current application, the same issues arise unless opposite electrodes are applied with a gap between them of about a millimeter and there are multiple conduction pathways. By reducing electrode contact area and inter-electrode gap, control is obtained of the anatomy of current flow, in particular where the skin acts as a dielectric conductor and breaks down its resistance under the electric field. This allows a surety of anatomical nerve or nerve branch targeting, impossible with the prevalent TENS electrode designs. Thus in this proposed device there are two distinct areas of the electrode design intended, one intended for more usual TENS counter irritation application, surrounding the other for a superficial nerve blockade. Further when skin penetration occurs the possible tendency for the TENS signal to “short circuit” (through the needle track in the epidermis) can be accommodated by affording switching of the signals between the inner sited electrode contacts and or those more peripherally located. By this means, the TENS current is relatively increased within the poorly conducting outer layers of the skin (specifically the epidermis) compared to the more dominant usual route whereby current tracks through sweat glands or hair follicles, down to the lower electrical resistance areas of the dermis. Increasing electrode area reduces the electrode to sub-dermal tissue resistance (which in absolute terms with normal sized electrodes is several orders of magnitude lower than a purely epidermal pathway resistance between electrodes). This is because far more sweat glands or hair follicles are covered by the electrode surface. As inter-electrode distance approaches or is less than twice epidermal thickness, conduction along the epidermal tissue becomes significant in proportion to the total current flow, and electrode area reduction further improves this efficiency at the expense of electrode effectiveness. However, the applied voltage needed to cause this current to flow increases, thus a balance is struck between area, voltage and inter-electrode gap such that both epidermal and dermal nerves are adequately exposed to the TENS signal. The arrangement proposed allows the un-myelinated “C” nerve fiber endings that lie within the epidermis and are part of the pain sensing mechanism better exposure to the depolarizing current. In the dermis it is believed that other nerve fiber types may in addition conduct pain signals, particularly the A-delta fibers. There are multiple low resistance routes for the current to take both within and below the dermis that avoid flow around nerve endings, limiting the distance between electrodes and electrode surface areas, increases the electrical potential or gradient in a small area. This both increases the probability of a nerve fiber in this region being depolarized or hyper-polarized and decreases the probability of current tracking into deeper tissues causing side effects such as muscle stimulation. Further, limitation of electrode area decreases electrode to muscle capacitance, thereby reducing signal conduction by capacitative coupling to this tissue and so reducing muscle stimulation.

In the usual application of TENS, acclimatization to the TENS current occurs in respect to discomfort caused by the current. This tolerance to the TENS current evolves in the patient over a short period of time (particularly with a continuous pulse signal as opposed to bursts of pulses at a low frequency). Thus, if the signal applied to the electrodes is built up slowly (over a few seconds) significantly higher signal strengths may be applied without causing significant discomfort. In the working device described here, electrical circuitry is so arranged that the TENS signal(s) builds up in intensity over a few seconds. In addition circuitry is provided that reduces the pulse width of the signal over a few seconds, again allowing a higher voltage to be applied without discomfort. This combination of electrode surface limitation, inter-electrode gap limitation, signal strength build up, pulse width control, outer electrode use and interplay with the inner electrodes as well as the timing and pattern (both in time and locality) of TENS signal application allow the use of an intense local TENS effect. This results in blocking and or distorting the usual pain signal formation and pattern of firing of the local nerves in the area of skin penetration. The overall effect is to cause miscommunication and misinterpretation of the pain signals at higher levels, in effect blocking or drastically reducing the sensation of pain experienced by the local tissue trauma. Each of these elements contributes in part to the overall effect, resulting in a relevant level of efficacy and comfort.

Nerve fiber parameters, frequency and wave form selection.

The possible frequency of action potentials of nerve fibers conducting pain signals may be measured or estimated and from this a projection of the ideal blocking signal parameters. Such information is available to a degree in standard texts of physiology. However, it is likely that there are both a range of fiber types and dimensions as well as their possible branching and networking arrangements that need to be addressed by the TENS signal. In some circumstances as nerve branches coalesce into the main trunk for example the frequency of transmitted impulses decline, in effect acting as a low pass filter. Further, it is known that there is not a simple one to one relationship between frequency of discharge, number of nerves involved or of nerve type and the perceived sensation of pain severity. These observations are consistent with a system with redundancy as described above. Electrically the skin acts as both a resistive and a capacitive barrier in parallel, thus increasing frequency naturally allows a lower resistance to current flow. Higher frequencies tend to direct the signal to muscle which itself has a relative high surface area capacitive quotient than nerve by a factor of 10 or more as well as having more total surface area. Thus, the strategy taken here is to present a sweep of pulses and amplitudes that fit a range of possibilities. TENS is usually applied as a core depolarizing pulse of the order of 50 to 200 micro-seconds long repetitively applied in a train, which may be broken up into blocks or bursts of pulses. A shorter pulse width (30 to 150 microseconds) appears to have an adequate level of effectiveness, though longer pulses are common in studies on nerve and are more effective in nerve stimulation. Applying these various signal forms through the electrode described produces the desired effect. If a prolonged exposure is desired (such as with a wound dressing) modulation of the plateau level of signal with a time constant of the order of a few tenths to hundredths of a second so helping ensure continuing efficacy.

Physical Vibration Application. The perception of the severity of pain is related amongst other things to the force or energy of injury (Differential ability of human cutaneous nociceptors to signal mechanical pain and to produce vasodilatation. Koltzenburg, Handwerker, J Neurosci 1994 March; 14 (3 Pt 2):1756-65). Vibration lowers the resistance to a needle entering the skin (in the same manner that wiggling the fingers in sand allows them to penetrate the sand more easily). In addition the provision of a vibration during the time of injection adds another distracting element to the nervous system. Thus a vibration generating device is added to the device enhancing its effectiveness.

1. (canceled)

2. (canceled)

3. (canceled)

4. (canceled)

5. (canceled)

6. (canceled)

7. (canceled)

8. (canceled)

9. (canceled)

10. (canceled)

11. (canceled)

12. (canceled)

13. (canceled)

14. (canceled)

15. (canceled)

16. (canceled)

17. (canceled)

18. (canceled)

19. (canceled)

20. (canceled)

21. (canceled)

22. (canceled)

23. (canceled)

24. (canceled)

25. An apparatus for reducing pain or hemorrhage, the apparatus comprising:
a current generating device configured to generate an electrical output Trans Epithelial Nerve Stimulating (TENS) current;
an array of electrodes electrically coupled to the current generating device and configured to be placed around an injection location on the skin of a patient; and
a current applying device configured to apply the TENS current sequentially to the electrodes.

26. The apparatus of claim 25, wherein each electrode has a surface area of up to five square millimeters.

27. The apparatus of claim 25 wherein the array of electrodes includes a first group and a second group, the current applying device being adapted to apply a first type of the TENS current to the first group and a second type of the TENS current to the second group.

28. The apparatus of claim 27 wherein said first group of electrodes is adapted to be disposed proximal to and circumscribe the injection location.

29. The apparatus of claim 27, wherein the first group of electrodes are spaced apart such that an edge of one electrode of the first group is less than three millimeters away from an edge of an adjacent electrode of the first group.

30. The apparatus of claim 29, wherein said gap is less than one millimeter.

31. The apparatus of claim 27 wherein the second group of electrodes forms a ring adapted to be disposed peripherally to the injection location.

32. The apparatus of claim 27 wherein each of the electrodes of the second group is controlled independently of each of the electrodes of the second group.

33. An apparatus for reducing pain or hemorrhage at an injection location on the skin of a patient, the device comprising:
a current generating device configured to generate first and second types of electrical output Trans Epithelial Nerve Stimulating (TENS) current;
an array of electrodes electrically coupled to the current generating device and configured to be placed around the location on the skin of the patient, the electrodes including a first group and a second group, the first group being configured to be disposed closer than the second group to the location on the skin of the patient; and
a current applying device configured to:
apply the first type of TENS current to the first group of electrodes; and
apply the second type of TENS current to the second group of electrodes.

34. The apparatus of claim 33 wherein the current applying device is adapted to switch TENS current between the electrodes both within the first group of electrodes and the second group of electrodes.

35. The apparatus of claim 33 wherein the current applying device is capable of activating the first group of electrodes in a pattern such that the injection location is effectively encircled by the TENS current.

36. The apparatus of claim 35 wherein the TENS current is applied in an encircling pattern having a plurality of pulses of varying power and varying duration.

37. The apparatus of claim 36 wherein the encircling pattern of the first group of electrodes is different from the encircling pattern of the second group of electrodes.

38. The apparatus of claim 36 wherein the varying duration is approximately between 50 to 200 milliseconds initially and decreases to between 30 and 100 milliseconds.

39. The apparatus of claim 33 wherein the first type of TENS current has a different timing than the second type of TENS current.

40. A method of reducing pain or hemorrhage of a superficial therapeutic injection or a superficial body tissue sampling, the method comprising the steps of:
placing an array of electrodes on the skin of a patient;
applying electrical output Trans Epithelial Nerve Stimulating (TENS) current to the electrodes; and
breaching the skin of the patient at a location within the array of electrodes.

41. The method of claim 40 wherein the skin breach occurs during the applying step.

42. The method of claim 40 wherein the array of electrodes includes a first group and a second group, the location of the skin breach being closer to the first group than to the second group.

43. The method of claim 42 wherein the applying step includes applying a different type of TENS current to the first group of electrodes than to the second group of electrodes.

44. The method of claim 43 wherein the second group of electrodes encircles the first group of electrodes.
